(19) United States
(12) Patent Application Publication
(10) Pub. No.: US 2002/0183574 A1
(43) Pub. Date: Dec. 5, 2002

(54) Hydrocarbon conversion process

(76) Inventors: John Thomas Dixon, Vanderbijlpark (ZA); Jacobus Johannes Cronje Grove, Beyerspark (ZA); Alta Ranwell, Vanderbijlpark (ZA)

Correspondence Address: KNOBBE MARTENS OLSON & BEAR LLP, 2040 MAIN STREET, FOURTEENTH FLOOR, IRVINE, CA, 92614, US

(21) Appl. No.: 10/155,869

(22) Filed: May 23, 2002

(30) Foreign Application Priority Data
Nov. 26, 1999 (ZA) 99/7340

(51) Int. Cl. C07C 2/06; C07C 2/08

(52) U.S. Cl. 585/511; 585/512; 585/513; 585/521; 585/522; 585/523; 585/525

(57) ABSTRACT

The invention provides a hydrocarbon conversion process for converting olefins to longer chain hydrocarbons, the process using a catalyst system including a non-nickel transition metal derived catalyst and one or more ionic liquids at a reaction temperature of between 10° C. and 130° C. and a reaction pressure of up to 100 Bar. The hydrocarbon conversion process may be oligomerisation and trimerisation.

### RELATED APPLICATION

This application is a continuation, under 35 U.S.C. §120, of International Patent Application No. PCT/ZA00/00233, filed on Nov. 24, 2000, under the Patent Cooperation Treaty (PCT), which was published by the International Bureau in English on May 31, 2001, which designates the U.S. and claims the benefit of South African Provisional Patent Application No. 99/7340, filed Nov. 26, 1999, and United States Provisional Patent Application No. 60/167,616, filed Nov. 26, 1999.

### BACKGROUND OF THE INVENTION

This invention relates to a hydrocarbon conversion process. More particularly, this invention relates to a hydrocarbon conversion process for the oligomerisation of olefins and derivatives thereof.

The oligomerisation of olefins, primarily α-olefins, with chromium catalysts has been extensively studied. More specifically, a number of chromium catalysts have been developed and used to trimerise olefins. In this regard, the trimerisation of ethylene to 1-hexene is significant since, in addition to its use as a specific chemical, 1-hexene is extensively used in polymerisation processes either as a monomer or co monomer. Furthermore, the trimeric products derived from longer chain olefins could be well utilized as synthetic lubricants (e.g. polyalphaolefins/PAO's), as well as various other applications such as drilling muds, and as feedstock to prepare detergents and plasticizers.

Known chromium-based processes for the trimerisation of ethylene to 1-hexene include:

a) A process disclosed in U.S. Pat. No. 4,668,838, in which olefins are trimerised by passing the olefin in contact with a catalyst comprising the reaction product of a chromium compound, an organoaluminium compound hydrolysed with a specific amount of water and a donor ligand selected from hydrocarbyl isonitriles, amines and ethers;

b) European Patent No. 0 416 304 discloses the trimerisation of olefins by chromium-containing compounds, such as, for example, chromium pyrrolides that are prepared by forming a mixture of a chromium salt, a metal amide and an electron pair donor solvent, such as, for example, an ether. These chromium catalysts can be used either unsupported or supported on an inorganic oxide;

c) European Patent No. 0 668 105 discloses a process to trimerise ethylene to 1-hexene comprising contacting ethylene with a stabilized catalyst system comprising a chromium source, a pyrrole-containing compound, a metal alkyl and an aromatic compound;

d) European Patent No. 0 706 983 discloses a process for preparing an α-olefin oligomer which comprises oligomerising an α-olefin in a saturated hydrocarbon solvent in the presence of a chromium-based catalyst system comprising a combination of at least a chromium compound, an amine or metal amide, an alkylaluminium compound and a non-coordinating Lewis acid-containing compound based on an element selected from groups IIIB, IVB, VB and VIB of the periodic table;

e) European Patent No. 0 699 648 discloses a process for producing 1-hexene which comprises trimerising ethylene in a 1-hexene solvent in the presence of a catalyst system obtainable by contacting in a 1-hexene solvent a chromium-containing compound, trialkylaluminium or dialkylaluminium hydride, a pyrrole compound or derivative thereof and a group 13 (III B) or group 14 (IV B) halogen compound; and

f) U.S. Pat. No. 5,811,618 discloses a process for the trimerisation of ethylene, said process comprising reacting ethylene, using a catalyst comprising an aluminoxane and a polydentate phosphine, arsenic and/or stibine coordination complex of a chromium salt, such that 1-hexene is formed.

Also, WO 98/47616 discloses that ionic liquids may be used as solvent and/or activator during the oligomerisation of olefins with a nickel complex as catalyst.

Heterogeneous catalysts systems are often selected over homogeneous catalyst systems due to relative ease of recovering and recycling such catalyst systems. However, the process of trimerisation of ethylene using chromium catalysts supported on an inorganic oxide has problems of insufficient activity, poor selectivity towards the intended trimeric product and leads to unsatisfactory levels of polyethylene by-product.

Trimerisation processes using unsupported chromium catalyst systems involve the use of unsaturated hydrocarbon and/or saturated hydrocarbon and/or aromatic solvents. Consequently, recovery of chromium compounds from the reactor effluent stream requires several sequential steps.

A need has thus been identified for a catalyst system capable of being easily recovered and recycled, without compromising the advantages associated with homogeneous systems.

Non-aqueous room-temperature ionic liquids are generally composed of large organic cations associated with inorganic or organic anions. They can be described as mixtures of salts that are liquid at temperatures below the individual melting points of the components.

Ionic liquids are either organic salts or mixtures consisting of at least one organic component, the most common organic salts used being alkylammonium, alkylphosphonium, N-alkylpyridinium, and N,N -dialkylimidazolium as cations, associated with inorganic or organic anions.

The two basic methods for the preparation of ionic liquids are metathesis of a halide salt with, for instance, a silver, group 1 metal or ammonium salt of the desired anion, and secondly acid-base neutralization reactions.

### DETAILED DESCRIPTION OF THE INVENTION

Thus, according to first aspect of the invention, there is provided a hydrocarbon conversion process using a catalyst system including a non-nickel transition metal derived catalyst and one or more ionic liquids.

According to a second aspect of the invention, there is provided an oligomerisation process using a catalyst system including a non-nickel transition metal derived catalyst and one or more ionic liquids.

The ionic liquids may be liquid at room temperature, i.e. at below 50° C., typically at below 30° C. Usually the ionic liquids are liquid at between 15° C. and 25° C.

The ionic liquids may be non-aqueous ionic liquids.

The ionic liquids may be used as a solvent/co-solvent or activator for the catalyst system of the invention.

It is believed that the physical properties of the ionic liquids which make them suitable for an oligomerisation catalyst system include them being:

(1) good solvents for a wide range of both inorganic and organic materials;

(2) composed of poorly coordinating ions, thereby having the potential to be highly polar yet non-coordinating solvents;

(3) immiscible with a number of organic solvents and providing a non-aqueous, polar alternative for two-phase systems; and

(4) non-volatile, hence being useable in high-vacuum systems.

It is further believed that the anions are essentially responsible for the chemical properties of ionic liquids, the most important of which, for coordination chemists, is the coordinating ability and/or reactivity of anions toward the metal centre of a complex. These properties are believed to depend on the nature of the anions themselves, such as the size and charge and also on the hardness of the metal centre, its oxidation state, and its surrounding ligands.

The ionic liquids of the process of the invention may include as cation an organic halide salt such as a quaternary ammonium salt, an imidazoline or alkylsubstituted imidazoline salt, a pyridinium or alkyl-substituted pyridinium salt, a sulfonium or alkylsulfonium salt, or a phosphonium or alkyl-substituted phosphonium salt, or other suitable anion leading to ionic liquids having the desired properties.

The ionic liquids of the process of the invention may include as anion compounds of the formula RₙMX₃₋ₙ, or RₘM₂X₆₋ₘ wherein

R is a C₁-CC₆ alkyl (or iso-alkyl) radical;

M is aluminium, gallium, boron or iron (III);

X is a halogen atom;

n is 0, 1, 2 or 3; and

m is 1, 2 or 3.

The metal component of the anion compounds, M, is typically aluminium and m and/or n is typically three; therefore, the anion is typically an alkylaluminium compound. One such alkylaluminium useful as an anion for the process of the invention is triethyaluminium.

In some embodiments of the process of the invention, the anion is an alkylaluminoxane such as methylaluminoxane, or a borate anion such as B(C₆F₅)₄⁻.

Miscibilities of the corresponding ionic liquids with organic reactants may be altered by varying the alkyl chain length of the cation, when appropriate.

The ionic liquids of the process of the invention may include as anion an alkylaluminoxane or iso-alkylaluminoxane. The alkylaluminoxane may be methylaluminoxane, also known as MAO.

The ionic liquids of the process of the invention may include as anion tin and germanium halides, BF₄⁻, SbF₆⁻, PF₆⁻, FSO₃⁻, CF₃SO₃⁻, (CF₃SO₂)₂N⁻, CF₃CO₂⁻, copper, ammonium or phosphonium chlorides.

The ionic liquids of the process of the invention may include as anion large, weakly coordinating anions such as tetraphenylborate (BPh₄⁻) and related anions such as fluorinated derivatives of BPh₄⁻.

The ionic liquids of the process of the invention may include B(p-C₆H₄F)₄⁻; B(C₆F₅)₄⁻, B(3,5-C₆H₃(CF₃)₂)₄⁻), and PH₃BCNBPh₃⁻.

Other useful anions include 1-carba-closo-dodecacarborate (CB₁₁H₁₂⁻) and related anions, 12-CB₁₁H₁₁Cl⁻, 12-CB₁₁H₁₁Br⁻, 12-CB₁₁H₁₁I⁻, 12-CB₁₁H₁₁(C₆F₅)⁻, 7,12-CB₁₁H₁₀, 7,12-CB₁₁H₁₀Br₂, 7,12-CB₁₁H₁₀I₂, 7, 8, 9, 10, 12-CB₁₁H₇C₁₅, and 7, 8, 9, 10, 11, 12-CB₁₁H₆C₁₆), as well as pentafluorooxotellurate (OTeF₅) and related anions.

Other useful anions include anions of the formula M(OTeF₅)ₙ^{m-}, wherein

M is selected from Ni, Cu, Zn and Pd;

n is either 4 or 6; and

m is either 1 or 2.

Yet further useful anions include PW₁₂O₄₀³⁻ and related anions, and HC(SO₂CF₃)₂⁻ together with related anions.

Even further useful anions include the fulleride ion C₆₀⁻, the relatively stable borate anion B(o-C₆H₄O₂)₂⁻ and the diborane anion H(1,8-(BMe₂)₂c₁₀H₆)⁻.

The non-nickel transition metal derived catalyst of the catalyst system may be a chromium derived catalyst.

The catalyst system of the process of the invention may be a trimerisation catalyst system.

The trimerisation catalyst system may include the ionic liquids as solvent or co-solvent and/or activator together with at least one of a chromium source and a pyrrole-containing compound. Typically the chromium source and pyrrole-containing compound are used in combination.

In addition to including at least one of the chromium source and the pyrrole-containing compound, the catalyst system may optionally include one or more of a metal alkyl, a halogen source, and an unsaturated hydrocarbon compound.

The chromium source of said catalyst system may consist of one or more organic and/or inorganic chromium compounds, with the chromium oxidation state ranging from 0 to 6.

These chromium compounds are suitably expressed by the general formula CrXₙ, wherein

X may be the same or different and represents an organic or inorganic radical, group or compound; and

n is an integer from 0 to 6.

Where at least one of the X's includes an organic radical, the organic radical may have from about 1 to 20 carbon atoms per radical. Typically such radical is selected from the group consisting of alkyl, alkoxy, ester, ketone and/or amido radicals.

Where at least one of the X's include an organic compound, then such organic compound may be selected from the group consisting of an amine compound, a phosphine compound, a phosphine oxide compound, a nitrosyl group, and an ether compound.

Where at least one of the X's include an inorganic radical, then such inorganic radical may be selected from the group consisting of halides, nitrates, and sulphates.

In specific examples of the trimerisation catalyst system, the chromium source may include one or more of chromium(III)acetylacetonate, chromium (III) acetate, chromium (III) pyrrolide, chromium (III) 2,2,6,6,tetramethylheptadionate, chromium (III) tris(2-ethylhexanoate), bis(N,N′-bis(trimethylsilyl)benzamidinato) chromium (III) chloride, trichlorotris(4-isopropylpyridine) chromium (III), trichloro (N,N,N′,N′,N″-pentamethyidiethylenetriamine) chromium (III) chloride, bis-(2-dimethylphosphino-ethyl)ethylphosphine chromium (III), (2-dimethylphosphino-ethyl)(3-dimethylphosphinopropyl)methylphosphine chromium (III) chromium (III) naphthenate, chromium (II) acetate, chromium (II) pyrrolides, chromium (II) bis(2-ethylhexanoate), and chromium (II) chloride.

The pyrrole-containing compound of the trimerisation catalyst system includes pyrrole and derivatives thereof.

The term “pyrrole” refers to hydrogen pyrrolide, i.e. C₄H₅N.

A “pyrrolide”, for purposes of this specification, is defined as a compound containing a 5-membered, nitrogen containing heterocycle.

The derivatives of pyrrole in the trimerisation catalyst system may include substituted pyrrolides, heteroleptic or homoleptic metal pyrrolide complexes, salts, and isotopes thereof.

The pyrrole-containing compounds of the trimerisation catalyst system may have from about 4 to about 20 carbon atoms per molecule.

The pyrrole-containing compounds of the trimerisation catalyst system may include hydrogen pyrrolide (pyrrole), sodium pyrrolide, lithium pyrrolide, potassium pyrrolide, caesium pyrrolide, chromium (III) pyrrolides, aluminium-diethyl-pyrrolide, ethyl-aluminium-dipyrrolide, aluminium-tripyrrolide, 2,5-dimethylpyrrole, 3,4-dimethylpyrrole, 3,4-dichloro pyrrole, indole, imidazole, 2-acyl-pyrrole, pyrrolidine, or the like.

The pyrrole-containing compounds of the trimerisation catalyst system may include derivatives of pyrrole having a hydrocarbon group bonded to the pyrrole ring.

These pyrrole-containing compounds having a hydrocarbon group bonded to the pyrrole ring are believed to enhance the activity and selectivity of the trimerisation catalyst system to desired products.

The pyrrole-containing compound can be either affirmatively added to the reaction, or generated in-situ.

The catalyst system of the process of the invention may optionally include one or more metal alkyls.

The metal alkyl may be any heteroleptic or homoleptic metal alkyl compound.

Typically, the alkyl ligands include any saturated aliphatic radical.

The alkyl ligands on the metal may be aliphatic or aromatic.

The metal alkyl may have any number of carbon atoms, however, typically less than about 20 carbon atoms per molecule.

The metal alkyl may be selected from the group including, alkyl aluminium compounds, hydrolysed alkyl aluminium compounds, aluminoxanes, alkyl boron compounds, alkyl magnesium compounds, alkyl zinc compounds, and alkyl lithium compounds.

Typically, the metal alkyl is an alkyl aluminium compound, hydrolysed alkyl aluminium compound, or an alkyl boron compound.

Usually the metal alkyl is an alkyl aluminium compound, selected from alkyl aluminium compounds expressed by the formula: AlXₙR₃₋ₙ, or RₙAlOR₃₋ₙ, wherein

n is an integer selected from 0, 1, 2 and 3;

R is an alkyl group; and

X is a halogen atom.

In specific embodiments, the alkyl aluminium compounds include trimethyl aluminium, triethyl aluminium, tripropyl aluminium, tributyl aluminium, triisobutyl aluminium, diethyl aluminium chloride, diethyl aluminium bromide, diethyl aluminium ethoxide, ethyl aluminium dichloride, ethyl aluminium sesquichloride, or the like, and mixtures thereof.

The use of said alkyl aluminium compound is believed to aid catalyst system activity and/or reduce the quantity of undesirably by-product polymer.

Furthermore, the catalyst system may optionally include one or more halogen sources. The optional halogen sources are believed to increase selectivity towards the formation of desired products.

The one or more halogen sources may be selected from any compound containing one or more halogen atoms.

The one or more halogen sources include compounds with the general formula of LₘRₙXₚ, wherein

L denotes a cation containing an element selected from those elements belonging to the 1-, 6-, 8-, 9-, 10-,11-, 13-, 15- and 17-Groups of the Periodic Table;

R may be any organic and/or inorganic radical;

X may be a halide, selected from the group consisting of fluoride, chloride, bromide, and iodide;

m is an integer ranging from 0 to 2;

p may be any integer greater than 0; and

n may be any integer, typically less than 100.

Where the R of the one or more halogen sources is an organic radical, typically the R is a cyclic or acyclic radical with about 1 to about 70 carbon atoms, usually having from about 1 to 20 carbon atoms per radical.

Where the R of the one or more halogen sources is an inorganic radical, typically the R is selected from those elements belonging to the 3-, 4-, 5-, 6-, 13-, 14- and 15-Groups of the Periodic Table.

The one or more halogen source may include one or more of the other components of the catalyst system including the chromium source, the metal alkyl and the electron pair donor solvent can contain and provide a halide to the reaction mixture.

In specific embodiments of the catalyst system the one or more halogen sources may be selected from the group including methylene chloride, chloroform, 1-bromobutane, 1,4-dibromobutane, 1,1,1-trichlorethane, 1,1,2,2-tetrachloroethane, hexachloroethane, hexachloro-benzene, 1,2,3-trichloro-cyclopropane, 1,2,3-trichlorocyclopentane, zirconium tetrachloride, titanium tetrachloride, silane tetrachloride, tin tertachloride, aluminium trichloride, boron trichloride, germanium tetrachloride, diethyl aluminium chloride, diethyl aluminium bromide, ethyl aluminium dichloride, 1-chloro-2-butene, allylchloride, tris (pentaflurophenyl)boron, potassium tetrakis (pentafluorophenyl)borate, potassium tetrafluoroaluminate, potassium tetrachloraluminate potassium tetrafluoroborate, tetrabutylammonium tetrafluoroborate, potassium tetrachloroborate, or the like.

The catalyst system may also include a hydrocarbon and/or hydrocarbon derived compound. It is believed that this compound may increase catalyst system activity and/or lower the viscosity of the ionic liquid phase.

The hydrocarbon and/or hydrocarbon derived compound may be any aromatic or aliphatic hydrocarbon compound.

The hydrocarbon and/or hydrocarbon derived compound may have any number of carbon compounds per molecule, but usually has less than 20 carbon atoms. The limitation to less than 20 carbon atoms is due to commercial availability and end use.

The hydrocarbon and/or hydrocarbon derived compound includes ethylene, 1-hexene, 1,3-butadiene, 1,4-cyclo-octadiene, benzene, toluene, ethylbenzene, xylene, fluorobenzene, cyclohexane, and the like.

It has been found that 1-hexene can be selectively produced by trimerisation of ethylene using a catalyst system comprising a combination of at least a chromium source and a pyrrole-containing compound in the presence of ionic liquids as solvent or co-solvent. In addition, the use of these ionic liquids as solvent for said chromium catalyst system, also selectively produces the trimeric products of longer chain olefins in high yields.

The process of the invention may be used for the trimerisation of ethylene to 1-hexene.

The process of the invention may be used for the trimerisation of longer chain olefins to their respective trimeric products.

The longer chain olefins may have from 2-20 carbon atoms.

The trimerisation of olefins having from 2 to 20 carbons, typically from 5 to 15 carbons, may produce a lubricant.

Where the olefins which are trimerised include 1-decene, then the lubricant may have a viscosity index of typically 130 or higher which corresponds to a high quality synthetic base oil like polyalphaolefin.

The longer chain olefins may include Fischer-Tropsch-derived olefins.

Typical process parameters include one or more of:

one or more chromium sources present in an amount of between 0.1 mmole per liter solvent and 10 mmole per liter solvent;

one or more pyrrole-containing compounds present in an amount of from 1 to 10 equivalents with respect to the chromium compounds;

one or more metal alkyls present in an amount of from 3 to 50 equivalents with respect to the chromium compounds;

one or more halogen sources present in an amount from 1 to 20 equivalents with respect to the chromium compounds;

a reaction temperature of between 10° C. and 130° C.; and

a reaction pressure of up to 100 Bar.

The process may be carried out in a batch reactor, in a continuous reactor, in a semi-batch reactor, or any other suitable reactor. For example, the process may be carried out in a fluid bed reactor, a slurry bed reactor, a fixed bed reactor, or the like.

It is believed that ionic liquids form biphasic systems with oligomerisation reagents and products, thereby permitting ease of continuous operation and separation and recycling of the catalyst system comparable to heterogeneous systems.

### EXAMPLES OF PERFORMING THE INVENTION

### Example 1

### Ethylene Trimerisation in an Ionic Liquid

All procedures were carried out under inert conditions, using pre-dried reagents.

The catalyst system was prepared as follows: 0.06 mmol of chromium(III) ethylhexanoate was combined with 0.18 mmol of 2,5-dimethylpyrrole and 0.9 mmol of triethylaluminium in cyclohexane. The catalyst complex was activated at room temperature for a period of two hours. A suitable ionic liquid [10 ml] (e.g. [Bmim][Et₃Al—Cl] was added to the activated complex, and the mixture was stirred for 15 minutes.

This solution, together with 40 ml cyclohexane, was charged to a pressure reactor, fitted with a mechanical stirrer. The reactor temperature was maintained at 100-120° C., while the ethylene pressure was kept at 50 bar. Thorough mixing was ensured by mixing speeds of 700 RPM's and higher.

Upon exit from the reactor, the product/solvent phase was decanted from the ionic liquid/catalyst phase, and the product/solvent phase was analysed by GC. The ionic liquid/catalyst phase could now be recycled.

A catalyst activity of 13500 g product/g Cr/hour was observed. The selectivity towards C₆ products was 64 mass %, while the selectivity observed towards 1-hexene formation was 83 mass %.

### Example 2

### 1-Decene Trimerisation in an Ionic Liquid

All procedures were carried out under inert conditions, using Schlenk apparatus and pre-dried reagents.

The catalyst system was prepared as follows: 0.06 moles of chromium(III) ethylhexanoate was combined with 0.18 moles of 2,5-dimethylpyrrole and 0.9 moles of triethylaluminium in cyclohexane. The catalyst complex was activated at room temperature for a period of two hours. 2 ml of a suitable ionic liquid such as [Bmim][Et3Al—Cl] was added to the activated complex, and the mixture was stirred for 15 minutes.

1-Decene (20 ml) was added to the activated catalyst system, and the mixture was stirred overnight by magnetic stirrer bar. The product/solvent phase was decanted from the ionic liquid/catalyst phase, and the product/solvent phase was analysed by high temperature GC. The GC indicated C₃₀ oligomers to be the main products, while the oligomer fraction contained three main isomers. The ionic liquid/catalyst phase could now potentially be recycled.

The product distribution obtained was as follows:

C₁₀-65%

C₂₀-3%

C₃₀-32%

The C₃₀ trimer fraction was isolated by short path distillation, and the viscosities at −40° C., 40° C. and 100° C. determined:

Viscosity@−40° C.: 1181.67 cSt

40° C.: 12.31 cSt

100° C.: 3.21 cSt

The viscosity index (VI) was calculated as 130, indicating that a polyalphaolefin quality lubricant was produced. NMR analyses of the product indicated mainly short chain branching explaining the good low temperature viscometrics.

### Example 3

### 1-Pentene Trimerisation in an Ionic Liquid

All procedures were carried out under inert conditions, using Schlenk apparatus and pre-dried reagents.

The catalyst system was prepared as follows: 0.06 mmol of chromium(III) ethylhexanoate was combined with 0.18 moles of 2,5-dimethylpyrrole and 0.9 moles of triethylaluminium in cyclohexane. The catalyst complex was activated at room temperature for a period of two hours. 2 ml of a suitable ionic liquid (e.g. [Bmim][Et₃Al—Cl] was added to the activated complex, and the mixture was stirred for 15 minutes.

1-Pentene (12 ml) was added to the activated catalyst system, and the mixture was stirred overnight by magnetic stirrer bar. The product/solvent phase was decanted from the ionic liquid/catalyst phase, and the product/solvent phase was analysed by high temperature GC. The GC indicated C₁₅ oligomers to be the main product, while the oligomer fraction contained three main isomers. The ionic liquid/catalyst phase could now potentially be recycled.

The product distribution obtained was as follows:

C₅-60%

C₁₀-3%

C₁₅-37%

NMR analysis to determine the isomer structures of the product indicated mainly short chain branching.

What is claimed is:

1. An olefin oligomerisation process for converting an olefin to a longer chain hydrocarbon, the process comprising the steps of:
providing a catalyst system comprising a non-nickel transition metal derived catalyst and an ionic liquid; and
converting an olefin to a longer chain hydrocarbon in the presence of the catalyst system at a reaction temperature of between 10° C. and 130° C. and a reaction pressure of up to 100 Bar.

2. The oligomerisation process as claimed in claim 1, wherein the step of converting the olefin to the longer chain hydrocarbon comprises trimerising the olefin.

3. The oligomerisation process as claimed in claim 1, wherein the ionic liquid comprises a non-aqueous ionic liquid.

4. The oligomerisation process as claimed in claim 1, wherein the ionic liquid comprises a poorly coordinating ion, such that the ionic liquid comprises a highly polar yet non-coordinating solvent.

5. The oligomerisation process as claimed in claim 1, wherein the catalyst system further comprises an organic solvent, and wherein the ionic liquid is immiscible with the organic solvent, such that the catalyst system comprises a non-aqueous, polar two-phase catalyst system.

6. The oligomerisation process as claimed in claim 1, wherein the ionic liquid comprises a cation selected from the group consisting of organic halides, imidazolines, alkylsubstituted imidazolines, pyridiniums, alkyl-substituted pyridiniums, sulfoniums, alkylsulfoniums, phosphoniums, and alkyl-substituted phosphoniums.

7. The oligomerisation process as claimed in claim 1, wherein the ionic liquid comprises an anion selected from the group consisting of:
anions of the formula RₙMX₃₋ₙ wherein:
R is a C₁-C₆ alkyl radical or iso-alkyl radical;
M is selected from the group consisting of aluminium, gallium, boron, and iron (III), wherein if M is aluminium, n is three;
X is a halogen atom; and
n is 0, 1, 2 or 3,
anions of the formula RₘM₂X₆₋ₘ wherein:
R is a C₁-C₆ alkyl radical or iso-alkyl radical;
M is selected from the group consisting of aluminium, gallium, boron, and iron (III), wherein if M is aluminium, m is three;
X is a halogen atom; and
m is 1, 2 or 3,
anions of the formula M(OTeF₅)ₙ^{m-} wherein:
M is selected from the group consisting of Ni, Cu, Zn, and Pd;
n is either 4 or 6; and
m is either 1 or 2,
B(p-C₆H₄F)₄⁻, B(C₆F₅)₄⁻, B(3,5-C₆H₃(CF₃)₂)₄⁻), PH₃BCNBPh₃⁻, 1-carba-closo-dodecacarborate (CB₁₁H₁₂⁻), derivatives of 1-carba-closo-dodecacarborate (CB₁₁H₁₂⁻), 12-CB₁₁H₁₁Cl⁻, 12-CB₁₁H₁₁Br, 12-CB₁₁H₁₁I⁻, 12-CB₁₁H₁₁(C₆F₅)⁻, 7,12-CB₁₁H₁₀, 7,12-CB₁₁H₁₀Br₂, 7,12-CB₁₁H₁₀I₂, 7,8,9,10,12-CB₁₁H₇C₁₅, 7,8,9,10,11,12-CB₁₁H₆C₁₆), pentafluorooxotellurate (OTeF₅), derivatives of pentafluorooxotellurate (OTeF₅) PW₁₂O₄₀³⁻, derivatives of PW₁₂O₄₀³⁻, HC(SO₂CF₃)₂⁻, derivatives of HC(SO₂CF₃)₂⁻, the fulleride ion C₆₀⁻, the borate anion B(o-C₆H₄O₂)₂⁻, the diborane anion H(1,8-(BMe₂)₂c₁₀H₆)⁻, alkylaluminium compounds, triethyaluminium, alkylaluminoxanes, ismethylaluminoxane, borate anions, B(C₆F₅)₄⁻, tin halides, germanium halides, BF₄⁻, SbF₆⁻, PF₆, FSO₃⁻, CF₃SO₃⁻, (CF₃SO₂)₂N⁻, CF₃CO₂⁻,copper chlorides, ammonium chlorides, phosphonium chlorides, tetraphenylborate (BPh₄⁻), and derivatives of tetraphenylborate (BPh₄⁻).

8. The oligomerisation process as claimed in claim 1, wherein the non-nickel transition metal derived catalyst comprises a chromium derived catalyst, the chromium derived catalyst comprising a chromium source present in an amount of between 0.1 mmole per liter solvent and 10 mmole per liter solvent.

9. A trimerisation process for the trimerisation of olefins having from 2 to 20 carbons, the process comprising the step of:
providing a trimerisation catalyst system which comprises an ionic liquid as a solvent, a co-solvent, or an activator; and a first component selected from the group consisting of a chromium source, a pyrrole-containing compound, and mixtures thereof, wherein the chromium source, if present, is present in an amount of between 0.1 mmole per liter solvent and 10 mmole per liter solvent, and wherein the pyrrole-containing compound, if present, is present in an amount of between 0.1 mmole per liter solvent and 100 mmole per liter solvent; and
trimerising an olefin in the presence of the trimerisation catalyst system at a reaction temperature of between 10° C. and 130° C. and a reaction pressure of up to 100 Bar.

10. The trimerisation process as claimed in claim 9, wherein the first component comprises a chromium source, and the catalyst system further comprises a second component selected from the group consisting of a metal alkyl, a halogen source, and an unsaturated hydrocarbon compound, wherein the metal alkyl, if present, is present in an amount of from 3 to 50 equivalents with respect to the chromium source, and wherein the halogen source, if present, is present in an amount from 1 to 20 equivalents with respect to the chromium source.

11. The trimerisation process as claimed in claim 10, wherein the chromium source is selected from the group consisting of:
a compound expressed by the general formula CrXₙ wherein:
n is an integer from 0 to 6;
X is the same or different and is selected from the group consisting of:
an organic radical having from 1 to 20 carbon atoms, wherein the organic radical is selected from the group consisting of alkyl, alkoxy, ester, ketone and amido,
an inorganic radical selected from the group consisting of halides, nitrates, and sulphates,
an amine compound,
a phosphine compound,
a phosphine oxide compound,
a nitrosyl group, and
an ether compound.

12. The trimerisation process as claimed in claim 9, wherein the chromium source comprises a chromium compound selected from the group consisting of chromium(III)acetylacetonate, chromium (III) acetate, chromium (III) pyrrolide, chromium (III) 2,2,6,6,tetramethylheptadionate, chromium (III) tris(2-ethylhexanoate), bis(N,N′-bis(trimethylsilyl)benzamidinato) chromium (III) chloride, trichlorotris(4-isopropylpyridine) chromium (III), trichloro (N,N,N′,N′,N″-pentamethyldiethylenetriamine) chromium (III) chloride, bis-(2-dimethylphosphino-ethyl)ethylphosphine chromium (III), (2-dimethylphosphino-ethyl)(3-dimethylphosphinopropyl)methylphosphine chromium (III) chromium (III) naphthenate, chromium (II) acetate, chromium (II) pyrrolides, chromium (II) bis(2-ethylhexanoate), and chromium (II) chloride.

13. The trimerisation process as claimed in claim 9, wherein the pyrrole-containing compound comprises pyrrole or a pyrrole derivative, wherein the pyrrole derivative is selected from the group consisting of substituted pyrrolides, heteroleptic metal pyrrolide complexes, homoleptic metal pyrrolide complexes, and salts and isotopes thereof.

14. The trimerisation process as claimed in claim 9, wherein the pyrrole-containing compound has from about 4 to about 20 carbon atoms, and is selected from the group consisting of hydrogen pyrrolide (pyrrole), sodium pyrrolide, lithium pyrrolide, potassium pyrrolide, caesium pyrrolide, chromium (III) pyrrolides, aluminium-diethyl-pyrrolide, ethyl-aluminium-dipyrrolide, aluminium-tripyrrolide, 2,5-dimethylpyrrole, 3,4-dimethylpyrrole, 3,4-dichloro pyrrole, indole, imidazole, 2-acyl-pyrrole, pyrrolidine, and pyrrole derivatives having a hydrocarbon group bonded to the pyrrole ring.

15. The trimerisation process as claimed in claim 9, wherein the catalyst system comprises a metal alkyl selected from the group consisting of heteroleptic metal alkyl compounds and homoleptic metal alkyl compounds; wherein the homoleptic metal alkyl compounds are selected from the group consisting of alkyl aluminium compounds, hydrolysed alkyl aluminium compounds, aluminoxanes, alkyl boron compounds, alkyl magnesium compounds, alkyl zinc compounds, alkyl lithium compounds, alkyl aluminium compounds having the formula:
AlXₙR₃₋ₙ wherein:
n is an integer selected from 0, 1, 2 and 3;
R is an alkyl group; and
X is a halogen atom, and
alkyl aluminium compounds having the formula:
RₙAlOR₃₋ₙ wherein:
n is an integer selected from 0, 1, 2 and 3;
R is an alkyl group; and
X is a halogen atom.

16. The trimerisation process as claimed in claim 9, wherein the catalyst system comprises a halogen source selected from the group consisting of:
compounds with the general formula of LₘRₙXₚ, wherein:
L comprises a cation comprising an element selected from the group consisting of elements belonging to the 1-Group of the Periodic Table, elements belonging to the 6-Group of the Periodic Table, elements belonging to the 8-Group of the Periodic Table, elements belonging to the 9-Group of the Periodic Table, elements belonging to the 10-Group of the Periodic Table, elements belonging to the 11-Group of the Periodic Table, elements belonging to the 13-Group of the Periodic Table, elements belonging to the 15-Group of the Periodic Table, and elements belonging to the 17-Group of the Periodic Table;
R is selected from the group consisting of an organic radical having from 1 to 70 carbon atoms and an inorganic radical, wherein the inorganic radical comprises an element selected from the group consisting of elements belonging to the 3-Group of the Periodic Table, elements belonging to the 4-Group of the Periodic Table, elements belonging to the 6-Group of the Periodic Table, elements belonging to the 13-Group of the Periodic Table, elements belonging to the 14-Group of the Periodic Table, and elements belonging to the 15-Groups of the Periodic Table;
X is a halide selected from the group consisting of fluoride, chloride, bromide, and iodide;
m is an integer ranging from 0 to 2;
p is any integer greater than 0; and
n is any integer,
the chromium source,
the metal alkyl, and
the ionic liquid.

17. The trimerisation process as claimed in claim 16, wherein n is an integer less than 100.

18. The trimerisation process as claimed in claim 16, wherein the halogen source is selected from the group consisting of methylene chloride, chloroform, 1-bromobutane, 1,4-dibromobutane, 1,1,1-trichlorethane, 1,1,2,2-tetrachloroethane, hexachloroethane, hexachloro-benzene, 1,2,3-trichloro-cyclopropane, 1,2,3-trichlorocyclopentane, zirconium tetrachloride, titanium tetrachloride, silane tetrachloride, tin tertachloride, aluminium trichloride, boron trichloride, germanium tetrachloride, diethyl aluminium chloride, diethyl aluminium bromide, ethyl aluminium dichloride, 1-chloro-2-butene, allylchloride, tris (pentaflurophenyl)boron, potassium tetrakis (pentafluorophenyl)borate, potassium tetrafluoroaluminate, potassium tetrachloraluminate potassium tetrafluoroborate, tetrabutylammonium tetrafluoroborate, and potassium tetrachloroborate.

19. The trimerisation process as claimed in claim 9, wherein the catalyst system comprises a compound selected from the group consisting of ethylene, 1-hexene, 1,3-butadiene, 1,4-cyclo-octadiene, benzene, toluene, ethylbenzene, xylene, fluorobenzene, and cyclohexane.

20. A process for producing a polyalphaolefin lubricant, the process comprising the steps of:
providing a trimerisation catalyst system comprising an ionic liquid as solvent, co-solvent, or activator; and a component selected from the group consisting of a chromium source, a pyrrole-containing compound, and mixtures thereof, wherein the chromium source, if present, is present in an amount of between 0.1 mmole per liter solvent and 10 mmole per liter solvent, and wherein the pyrrole-containing compound, if present, is present in an amount of between 0.1 mmole per liter solvent and 100 mmole per liter solvent; and
trimerising an olefin having from 2 to 20 carbons in the presence of the trimerisation catalyst at a reaction temperature of between 10° C. and 130° C. and a reaction pressure of up to 100 Bar, thereby producing a polyalphaolefin lubricant.

21. A process as claimed in claim 20, wherein the olefin has from 5 to 15 carbons.

22. A process as claimed in claim 20, wherein the olefin comprises 1-decene and wherein the lubricant has a viscosity index of at least 130.

23. A process as claimed in claim 20, wherein the olefin comprises a Fischer-Tropsch olefin.
